(19) United States
(12) Patent Application Publication
(10) Pub. No.: US 2026/0364077 A1
(43) Pub. Date: Nov. 19, 2026

(54) Method for Predicting Success in Test Subject of Cancer Treatment Drug Which Regulates Splicing Function

(71) Applicant: NATIONAL CANCER CENTER, Tokyo (JP)

(72) Inventors: Shinji KOHSAKA, Tokyo (JP); Hiroyuki MANO, Tokyo (JP); Akinobu HAMADA, Tokyo (JP)

(73) Assignee: NATIONAL CANCER CENTER, Tokyo (JP)

(21) Appl. No.: 19/131,937

(22) PCT Filed: Nov. 16, 2023
(86) PCT No.: PCT/JP2023/041306

§ 371 Date: May 21, 2025

(30) Foreign Application Priority Data
Nov. 24, 2022 (JP) 2022-187152

(51) Int. Cl. A61K 31/519; A61K 31/351; A61K 31/365; A61K 31/404; A61K 31/415; A61K 31/496; A61K 31/506; A61K 31/635; A61P 35/00; C12Q 1/6886

(52) U.S. Cl. CPC A61K 31/519; A61K 31/351; A61K 31/365; A61K 31/404; A61K 31/415; A61K 31/496; A61K 31/506; A61K 31/635; A61P 35/00; C12Q 1/6886; C12Q 2600/156

(57) ABSTRACT

A method for predicting the success in a test subject of a cancer treatment drug which regulates splicing function, said method involving a step for determining the presence/absence of a mutation in a homologous recombination repair-related gene by using a specimen collected from the test subject, and a step for predicting the success in the test subject of the cancer treatment drug for regulating splicing function on the basis of the presence/absence of said mutation.

### TECHNICAL FIELD

The present disclosure relates to a method for predicting the efficacy of a cancer therapeutic that regulates splicing function in a subject.

### BACKGROUND ART

When a mutation or a change in expression level occurs in a biomolecule related to splicing, proper production of mature mRNA from pre-mRNA cannot be achieved during RNA splicing. This results in decreased protein levels or the expression of an abnormal protein, which can ultimately lead to cell carcinogenesis. A mutation in a biomolecule related to splicing that causes cell carcinogenesis includes a mutation in a gene encoding a splicing factor such as SF3B1, SRSF2, or U2AF1.

The use of a drug that regulates splicing function as a cancer therapeutic is expected across a wide range of cancer types, including myeloid tumors such as hematologic tumors, lymphoid tumors, and solid tumors. For example, regarding sulfonamide-based small molecule drugs such as Indisulam (also known as E7070). E7820, and NSC339004 (chloroquinoxaline sulfonamide, CQS), which were known as a candidate compound for a cancer therapeutic against blood cancer and solid cancer, it has been found that their promotion of the proteasomal degradation of RBM39 (also known as CAPERα), a protein that promotes splicing in cancer cells, is their mechanism of action (Non Patent Literature 1).

A homologous recombination repair-related gene (hereinafter also referred to as “HR repair-related gene”) is a gene that encodes a protein responsible for repairing a broken DNA double strand in a manner that does not involve crossover. It is known that a mutation in a homologous recombination repair gene is prevalent in cancer, and a DNA-damaging agent, which is a type of cancer therapeutic, exhibits high treatment efficacy on a cancer with a mutation in this gene. For example, Patent Literature 1 describes that inhibition of the base excision repair (BER) pathway using an inhibitor targeting a component of BER is selectively lethal to a cell deficient in HR-dependent DNA double-strand break repair. In Patent Literature 1, a cancer cell with a phenotype deficient in BRCA1 or BRCA2 is given as a specific example of a cell deficient in HR-dependent DNA double-strand break repair.

### CITATION LIST

### Patent Literature

Patent Literature 1: WO2005/053662
Patent Literature 2: WO2001/056607
Patent Literature 3: WO95/007276
Patent Literature 4: WO2007/043621
Patent Literature 5: WO2004/011459
Patent Literature 6: WO2018/360559
Patent Literature 7: WO2014/100719
Patent Literature 8: WO2017/212385
Patent Literature 9: WO2020/152557
Patent Literature 10: WO2017/032840

### Non Patent Literature

Non Patent Literature 1: Taisuke Uehara et al., “Selective degradation of splicing factor CAPERα by anticancer sulfonamides.”. Nat. Chem. Biol. 13, 675-680 (2017).
Non Patent Literature 2: Kotake Y. et al., “Splicing factor SF3b as a target of the antitumor natural product pladienolide.”, Nat. Chem. Biol. 3, 570-575 (2017).
Non Patent Literature 3: Arun K. Ghosh and Zhi-Hua Chen, “Enantioselective Syntheses of FR901464 and Spliceostatin A: Potent Inhibitors of Spliceosome.”, Org. Lett. 15, 5088-5091 (2013).
Non Patent Literature 4: Kaida D. et al., “Spliceostatin A targets SF3b and inhibits both splicing and nuclear retention of pre-mRNA.”, Nat. Chem. Biol. 3, 576-583 (2007).
Non Patent Literature 5: Andrew Fedoriw et al., “Anti-tumor Activity of the Type I PRMT Inhibitor, GSK3368715, Synergizes with PRMT5 Inhibition through MTAP Loss.”, Cancer Cell 36, 100-114.E25 (2019).

### SUMMARY OF INVENTION

### Technical Problem

To date, the relationship between a mutation in a homologous recombination repair-related gene and the efficacy of a cancer therapeutic that regulates splicing function has not been known.

The present disclosure aims to provide a method for predicting the efficacy of a cancer therapeutic that regulates splicing function, and a method for selecting a patient who is expected to benefit from a cancer therapeutic that regulates splicing function.

### Solution to Problem

The present inventors examined the relationship between the efficacy of E7820 and gene mutations in a patient-derived xenograft (PDX) model mouse. They found that E7820 exhibited a high tumor reduction effect in a model mouse transplanted with a tumor having a mutation in a homologous recombination repair-related gene.

Furthermore, the present inventors found that a cell line having a mutation in a homologous recombination repair-related gene exhibited high drug sensitivity to various cancer therapeutics that regulate splicing function, not limited to E7820, and thus completed the present disclosure.

That is, the present disclosure relates to the following [1] to [8].

[1]A method for predicting efficacy of a cancer therapeutic that regulates splicing function in a subject, comprising:
a step of determining the presence or absence of a mutation in a homologous recombination repair-related gene by using a sample taken from the subject, and
a step of predicting efficacy of the cancer therapeutic that regulates splicing function in the subject based on the presence or absence of the mutation.
   [2]A method for selecting a patient who is expected to benefit from a cancer therapeutic that regulates splicing function, comprising:
a step of determining the presence or absence of a mutation in a homologous recombination repair-related gene by using a sample taken from a subject, and
a step of providing an indication that, if one or more of the genes have the mutation, the subject is a patient to whom the cancer therapeutic that regulates splicing function should be administered.
   [3] The method according to [1] or [2], wherein the cancer therapeutic that regulates splicing function comprises one or more compounds selected from the group consisting of a RBM39 degradation inducer, a SF3b complex inhibitor, a PRMT5 inhibitor, and a type I PRMT inhibitor.
   [4] The method according to [1] or [2], wherein the cancer therapeutic that regulates splicing function comprises one or more compounds selected from the group consisting of a RBM39 degradation inducer, a SF3b complex inhibitor, and a PRMT5 inhibitor.
   [5] The method according to [1] or [2], wherein the cancer therapeutic that regulates splicing function comprises one or more compounds selected from the group consisting of E7820, E7070, NSC339004, pladienolide B, E7107, spliceostatin A, GSK3326596, PF-06939999, JNJ-64619178, and GSK3368715.
   [6] The method according to any one of [1] to [5], wherein the homologous recombination repair-related gene comprises one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2.
   [7] The method according to any one of [1] to [5], wherein the homologous recombination repair-related gene comprises one or more genes selected from the group consisting of BRCA1, BRCA2, ATM, ATR, and BAP1.
   [8] The method according to any one of [1] to [5], wherein the homologous recombination repair-related gene comprises one or more genes selected from the group consisting of BRCA1 and BRCA2.

### Advantageous Effects of Invention

According to the present disclosure, a method for predicting the efficacy of a cancer therapeutic that regulates splicing function, and a method for selecting a patient who is expected to benefit from a cancer therapeutic that regulates splicing function can be provided.

### BRIEF DESCRIPTION OF DRAWINGS

FIG. 1 shows the relationship between the therapeutic effect of the cancer therapeutic E7820 that regulates splicing function and gene mutations in the PDX mouse model in Example 1.

FIG. 2 shows the results of evaluating the sensitivity of cultured cell lines to the cancer therapeutic E7820 that regulates splicing function, according to the presence or absence of mutations and knockouts in homologous recombination repair-related genes in Example 2.

FIG. 3 shows the results of evaluating the sensitivity of cultured cell lines to the cancer therapeutic Indisulam (E7070) that regulates splicing function, according to the presence or absence of mutations and knockouts in homologous recombination repair-related genes in Example 2.

FIG. 4 shows the results of evaluating the sensitivity of cultured cell lines to the cancer therapeutic pladienolide B that regulates splicing function, according to the presence or absence of mutations and knockouts in homologous recombination repair-related genes in Example 2.

FIG. 5 shows the results of evaluating the sensitivity of cultured cell lines to the cancer therapeutic JNJ-64619178 that regulates splicing function, according to the presence or absence of mutations and knockouts in homologous recombination repair-related genes in Example 2.

FIG. 6 shows the results of evaluating the sensitivity of cultured cell lines to olaparib, according to the presence or absence of mutations and knockouts in homologous recombination repair-related genes in Example 2.

FIG. 7 shows the results of evaluating the sensitivity of cultured cell lines to cisplatin, according to the presence or absence of mutations and knockouts in homologous recombination repair-related genes in Example 2.

FIG. 8 shows the results of evaluating the sensitivity of cultured cell lines to the cancer therapeutic that regulates splicing function, according to the presence or absence of knockouts and knock-ins in homologous recombination repair-related genes in Example 3.

FIG. 9 shows the results of evaluating the induction of DNA double-strand breaks by the cancer therapeutic that regulates splicing function, according to the presence or absence of knockouts and knock-ins in homologous recombination repair-related genes in Example 4.

### DESCRIPTION OF EMBODIMENTS

Hereinafter, embodiments for carrying out the present disclosure will be described in detail, but the present disclosure is not limited to the following embodiments.

One aspect of the present disclosure is a method for predicting the efficacy of a cancer therapeutic that regulates splicing function in a subject, comprising a step of determining the presence or absence of a mutation in a homologous recombination repair-related gene by using a sample taken from a subject, and a step of predicting the efficacy of the cancer therapeutic that regulates splicing function in the subject based on the presence or absence of the mutation. In one embodiment, this aspect may be a method for assisting in predicting the efficacy of a cancer therapeutic that regulates splicing function in a subject, comprising these steps. In one embodiment, this aspect may also be a method for obtaining data for predicting the efficacy of a cancer therapeutic that regulates splicing function in a subject, comprising these steps. In one embodiment, this aspect may also be a method for treating cancer in a subject, comprising a step of determining the presence or absence of a mutation in a homologous recombination repair-related gene by using a sample taken from a subject, and a step of administering a cancer therapeutic that regulates splicing function to the subject if one or more of the genes have the mutation.

In the present disclosure, a cancer therapeutic that regulates splicing function refers to a drug that exhibits antitumor activity by inhibiting, degrading, or suppressing the expression (hereinafter also referred to as “regulation, etc.”) of a biomolecule or molecular complex that promotes splicing in cancer cells. The biomolecule or molecular complex includes a splicing factor (SF), an mRNA precursor-binding protein, an mRNA precursor-binding nucleic acid, and an mRNA precursor-binding protein-nucleic acid complex. The splicing factor may be a mutated splicing factor (mutant splicing factor). It is known that when a mutation occurs in a splicing factor, proper removal of introns and proper joining of exons from the mRNA precursor (pre-mRNA) cannot be achieved, leading to the inability to produce proper mature mRNA, which can result in cell carcinogenesis.

Examples of splicing factors likely to undergo mutations, that are subject to regulation, etc., by a cancer therapeutic that regulates splicing function, include SF3B1 (Splicing Factor 3B Subunit 1), SRSF2 (Serine and Arginine Rich Splicing Factor 2), U2AF1 (U2 Small Nuclear RNA Auxiliary Factor 1), ZRSR2 (Zinc Finger CCHC-type, RNA Binding Motif and Serine/Arginine Rich 2), RBM10 (RNA-Binding Motif 10), and FUBP1 (Far Upstream Element-Binding Protein 1). The target of regulation, etc., by a cancer therapeutic that regulates splicing function may also be a complex containing a mutant splicing factor, such as an SF3b complex containing a mutant splicing factor.

In addition to the aforementioned splicing factors, examples of biomolecules or molecular complexes likely to promote splicing in cancer cells, that are subject to regulation, etc., by a cancer therapeutic that regulates splicing function, include RBM39 (RNA-binding Motif Protein 39, also known as CAPERα, HCC1, FSAP59, and RNPC2), the PRMT5/MEP50 complex (Protein Arginine Methyltransferase 5/Methylosome Protein 50 complex), PRMT5, type I PRMT, CLK (Cdc2-like Kinase), SRPK (SR Protein Kinase), DYRK (Dual-specificity Tyrosine-(Y)-Phosphorylation-regulated Kinase), SR protein, and hnRNP (heterogeneous nuclear RNA).

The cancer therapeutic that regulates splicing function according to the present disclosure may include, for example, one or more compounds selected from the group consisting of a RBM39 degradation inducer, a SF3b complex inhibitor, a PRMT5 inhibitor, a type I PRMT inhibitor, a PRMT5/MEP50 inhibitor, a CLK inhibitor, and a SRPK inhibitor. It may include one or more compounds selected from the group consisting of a RBM39 degradation inducer, a SF3b complex inhibitor, a PRMT5 inhibitor, and a type I PRMT inhibitor. It may also include one or more compounds selected from the group consisting of a RBM39 degradation inducer, a SF3b complex inhibitor, and a PRMT5 inhibitor. Furthermore, the cancer therapeutic that regulates splicing function according to the present disclosure may be, for example, a RBM39 degradation inducer, a SF3b complex inhibitor, a PRMT5 inhibitor, or a type I PRMT inhibitor. It may be a RBM39 degradation inducer, a SF3b complex inhibitor, or a PRMT5 inhibitor. It may also be a RBM39 degradation inducer.

A RBM39 degradation inducer induces the degradation of the protein RBM39, which promotes splicing in cancer cells. Typically, it mediates the binding between the E3 ubiquitin ligase DCAF15 (DDB1 and CUL4 associated factor 15)-CRL4 (Cullin-RING Finger Ligase 4) complex (CRL^{DCAF15}) as molecular glue, thereby promoting proteasomal degradation of RBM39 through polyubiquitination.

A SF3b complex inhibitor exhibits antitumor effects by inhibiting the binding between the SF3b (Splicing Factor 3b) complex containing a mutant splicing factor and the intron region of the mRNA precursor.

A PRMT5 inhibitor, a type I PRMT inhibitor, a PRMT5/MEP50 inhibitor, a CLK inhibitor, and an SRPK inhibitor exhibit antitumor effects by directly inhibiting the phosphorylation of a biomolecule or molecular complex that promotes splicing in cancer cells by a kinase, or by indirectly inhibiting the methylation of arginine residues of the kinase, thereby regulating splicing in cancer cells.

The cancer therapeutic that regulates splicing function according to the present disclosure may include, for example, one or more compounds selected from the group consisting of E7820, E7070, NSC339004, pladienolide B, E7107, spliceostatin A, GSK3326596, PF-06939999, JNJ-64619178, and GSK3368715. It may include one or more compounds selected from the group consisting of E7820, E7070, GSK3326596, PF-06939999, JNJ-64619178, and GSK3368715. It may also include one or more compounds selected from the group consisting of E7820 and E7070. In one embodiment, the cancer therapeutic that regulates splicing function may be E7820 or E7070, or it may be E7820.

E7820 is a compound with the chemical name N-(3-cyano-4-methyl-1H-indol-7-yl)-3-cyanobenzenesulfonamide, and its structure and synthesis method are described in Patent Literature 2. E7820 is a sulfonamide-based small molecule drug that exhibits antitumor effects as an RBM39 degradation inducer (Non Patent Literature 1).

E7070 (also known as Indisulam) is a compound with the chemical name N-(3-chloro-1H-indol-7-yl)-4-sulfamoylbenzenesulfonamide, and its structure and synthesis method are described in Patent Literature 3. E7070 is a sulfonamide-based small molecule drug that exhibits antitumor effects as an RBM39 degradation inducer (Non Patent Literature 1).

NSC339004 (also known as chloroquinoxaline sulfonamide, CQS) is a compound with the chemical name 4-amino-N-(5-chloroquinoxalin-2-yl)benzenesulfonamide. NSC339004 is a sulfonamide-based small molecule drug that exhibits antitumor effects as an RBM39 degradation inducer (Non Patent Literature 1).

Pladienolide B is a compound with the chemical name (4R,7R,8S,9E,11S,12S)-8-(acetyloxy)-4,7-dihydroxy-12-[(1E,3E,5S)-6-[(2R,3R)-3-[(1R,2S)-2-hydroxy-1-methylbutyl]-2-oxiranyl]-1,5-dimet hyl-1,3-oxacyclododeca-9-en-2-one, and its structure and synthesis method are described in Patent Literature 4. Pladienolide B exhibits antitumor effects as an SF3b complex inhibitor (Non Patent Literature 2).

E7107 is a compound with the chemical name [(2S,3S,4E,6S,7R,10R)-7,10-dihydroxy-2-[(2E,4E,6R)-6-hydroxy-7-[(2R,3R)-3-[(2R,3S)-3-hydroxypentan-2-yl]oxiran-2-yl]-6-methylhepta-2,4-dien-2-yl]-3,7-dimethyl-12-oxo-1-oxacyclododeca-4-en-6-yl]-4-cy cloheptylpiperazine-1-carboxylate or (8E,12E,14E)-7-[(4-cycloheptylpiperazin-1-yl)carbonyl]oxy-3,6,16,21-tetrahydroxy-6,10,12,16,20-pentamethyl-18,19-epoxytricosa-8,12,14-tr ien-11-olide, and its structure and synthesis method are described in Patent Literature 5 and 6. E7107 exhibits antitumor effects as an SF3b complex inhibitor (Non Patent Literature 2).

Spliceostatin A is a compound with the chemical name [(2S,3Z)-4-[[(2R,3R,5S,6S)-6-[(2E,4E)-5-[(3R,4R,5R,7S)-4-hydroxy-7-methoxy-7-methyl-1,6-dioxaspiro[2.5]octan-5-yl]-3-methylpenta-2,4-dien-1-yl]-2,5-dimethyloxan-3-yl]carbamoyl]but-3-en-2-yl]acetate, and its structure and synthesis method are described in Non Patent Literature 3. Spliceostatin A exhibits antitumor effects as an SF3b complex inhibitor (Non Patent Literature 4).

GSK3326596 (also known as EPZ015938) is a compound with the chemical name 6-[(1-acetylpiperidin-4-yl)amino]-N-[(2S)-3-(3,4-dihydro-1H-isoquinol in-2-yl)-2-hydroxypropyl]pyrimidine-4-carboxamide, and its structure and synthesis method are described in Patent Literature 7. GSK3326596 exhibits antitumor effects as a PRMT5/MEP50 inhibitor (Patent Literature 7).

PF-06939999 is a compound with the chemical name (1S,2S,3S,5R)-3-[(6-[difluoromethyl]-5-fluoro-1,2,3,4-tetrahydroisoqui nolin-8-yl)oxy]-5-(4-methyl-7H-pyrrolo[2,3-d]pyrimidin-7-yl)cyclopen tane-1,2-diol, and its structure and synthesis method are described in Patent Literature 8 and 9. PF-06939999 exhibits antitumor effects as a PRMT5 inhibitor (Patent Literature 8).

JNJ-64619178 (also known as Onametostat) is a compound with the chemical name (1S,2R,3S,5R)-3-[2-(2-amino-3-bromoquinolin-7-yl)ethyl]-5-(4-amino-7H-pyrrolo[2,3-d]pyrimidin-7-yl)cyclopentane-1,2-diol, and its structure and synthesis method are described in Patent Literature 10. JNJ-64619178 exhibits antitumor effects as a PRMT5 inhibitor (Patent Literature 10).

The structure and synthesis method of GSK3368715 (also known as EPZ019997) are described in Non Patent Literature 5. GSK3368715 exhibits antitumor effects as a type I PRMT inhibitor (Non Patent Literature 5).

In the present disclosure, a subject may be a cancer patient or a human subject who may have cancer.

In the present disclosure, a sample is not particularly limited as long as it can be collected from a subject and can potentially be used to determine the presence or absence of a mutation in a homologous recombination repair-related gene of the subject. For example, it may be whole blood, plasma, serum, bone marrow fluid, saliva, sputum, nipple discharge, urine, sweat, or pancreatic fluid, or it may be a biological sample obtained by scraping the uterine mucosa or collecting tumor tissue or lymph nodes.

In the present disclosure, a homologous recombination repair-related gene refers to a gene that encodes a group of proteins that directly or indirectly promote the repair of broken DNA double strands in a manner that does not involve crossover, i.e., homologous recombination repair. Examples of homologous recombination repair-related genes include BRCA1 (Breast Cancer Susceptibility Gene 1), BRCA2 (Breast Cancer Susceptibility Gene 2), ATM (Ataxia Telangiectasia Mutated), ATR (Ataxia Telangiectasia and Rad3-related), BAP1 (BRCA1-associated Protein 1), BLM (Bloom Syndrome), CHEK1 (Checkpoint Kinase 1), CHEK2 (Checkpoint Kinase 2), FANCA (Fanconi Anemia Complementation Group A), FANCM (Fanconi Anemia Complementation Group M), MRE11A (Meiotic Recombination 11 Homolog A), PALB2 (Partner and Localizer of BRCA2), RAD51 (RAD51 Recombinase), RAD54L (RAD54 Like), BRIP1 (BRCA1 Interacting Protein), CDK12 (Cyclin-dependent Kinase 12), and HDAC2 (Histone Deacetylase 2).

The homologous recombination repair-related gene according to the present disclosure may include one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2. It may include one or more genes selected from the group consisting of BRCA1, BRCA2, ATM, ATR, and BAP1. It may also include one or more genes selected from the group consisting of BRCA1 and BRCA2. Furthermore, the homologous recombination repair-related gene according to the present disclosure may be one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2. It may be one or more genes selected from the group consisting of BRCA1, BRCA2, ATM, ATR, and BAP1. It may be one or more genes selected from the group consisting of BRCA1 and BRCA2. It may also be BRCA1 or BRCA2.

The type of mutation that occurs in a homologous recombination repair-related gene according to the present disclosure is not particularly limited and may be, for example, a missense mutation, an inframe insertion, an inframe deletion, a frameshift mutation, a splice site mutation, or a nonsense mutation.

The method for determining the presence or absence of a mutation in a homologous recombination repair-related gene according to the present disclosure is not particularly limited as long as it can determine the presence or absence of a mutation in a homologous recombination repair-related gene. For example, it may be a method using a sequencer such as a next-generation sequencer (NGS), a method for analyzing a gene amplified by polymerase chain reaction (PCR) or reverse transcription polymerase chain reaction (RT-PCR), an Invader assay, a fluorescence in situ hybridization (FISH) method, or an immunostaining method. Methods for analyzing a gene amplified by PCR may include, for example, PCR-restriction fragment length polymorphism (PCR-RFLP), PCR-single strand conformation polymorphism (PCR-SSCP), TaqMan assay, multiplex ligation-dependent probe amplification (MLPA), or digital PCR.

Methods for analyzing a gene amplified by RT-PCR may include, for example, DNA microarray. These methods for determining the presence or absence of a mutation in a homologous recombination repair-related gene can determine the presence or absence of a mutation in a homologous recombination repair-related gene by using devices and reagents commonly used by those skilled in the art in methods commonly used by those skilled in the art.

The step of predicting the efficacy of a cancer therapeutic that regulates splicing function in a subject according to one aspect is performed based on the presence or absence of a mutation in a homologous recombination repair-related gene. That is, if a sample taken from a subject has a mutation in a homologous recombination repair-related gene, it can be predicted that the cancer therapeutic that regulates splicing function will have high efficacy in the subject. As a criterion for predicting high efficacy of the cancer therapeutic that regulates splicing function, for example, it may be predicted to have high efficacy if the number of genes with mutations among the genes examined for the presence or absence of mutations is above a reference value, and the reference value is not limited and may be one or two, for example. Another criterion for predicting high efficacy of the cancer therapeutic that regulates splicing function may be, for example, if the proportion of genes with mutations among the genes examined for the presence or absence of mutations is above a reference percentage, and the reference percentage is not limited and may be 5%, 10%, 15%, or 20%, for example.

Another aspect of the present disclosure is a method for selecting a patient who is expected to benefit from a cancer therapeutic that regulates splicing function, comprising a step of determining the presence or absence of a mutation in a homologous recombination repair-related gene by using a sample taken from a subject, and a step of providing an indication that, if the gene has the mutation, the subject is a patient to whom the cancer therapeutic that regulates splicing function should be administered.

The selection of a patient who is expected to benefit from a cancer therapeutic that regulates splicing function according to one aspect is performed based on the presence or absence of a mutation in a homologous recombination repair-related gene. That is, by determining the presence or absence of a mutation using a sample taken from a subject, an indication is provided that a subject determined to have a mutation in a homologous recombination repair-related gene is a patient to whom the cancer therapeutic that regulates splicing function should be administered, thereby selecting a patient who is expected to benefit from the cancer therapeutic that regulates splicing function. As a criterion for providing an indication that the subject is a patient to whom the cancer therapeutic that regulates splicing function should be administered, for example, it may be provided if the number of genes with mutations among the genes examined for the presence or absence of mutations is above a reference value, and the reference value is not limited and may be one or two, for example. Another criterion for providing an indication that the subject is a patient to whom the cancer therapeutic that regulates splicing function should be administered may be, for example, if the proportion of genes with mutations among the genes examined for the presence or absence of mutations is above a reference percentage, and the reference percentage is not limited and may be 5%, 10%, 15%, or 20%, for example.

Without wishing to be bound by any theory, one possible reason why the method according to the present disclosure can predict the efficacy of a cancer therapeutic that regulates splicing function in a subject or select a patient who is expected to benefit from a cancer therapeutic that regulates splicing function is that the cancer therapeutic that regulates splicing function induces DNA double-strand breaks.

### EXAMPLES

Hereinafter, the present invention will be described more specifically based on examples. However, the present invention is not limited to the following examples.

### Example 1: Relationship Between the Therapeutic Effect of a Cancer Therapeutic that Regulates Splicing Function and Gene Mutations in a PDX Mouse Model

In 30 cases of patient-derived xenograft (PDX) mouse models, the therapeutic effect of the cancer therapeutic E7820 that regulates splicing function and the presence or absence of various gene mutations were evaluated.

### [1-1: Preparation of PDX Model Mice]

PDX model mice were prepared by transplanting 2-3 mm square PDX tumor fragments subcutaneously into the back of test animals and allowing them to grow. The test animals used were NOD.Cg-Prkdcscidll2rgtmlSug/ShiJic (NOG mice), female, and 6 weeks old or older. Tumor tissues from 30 patients, including 9 cases of cholangiocarcinoma, 9 cases of pancreatic cancer, 6 cases of gastric cancer, 3 cases of endometrial cancer, and 3 cases of uterine sarcoma, were used as tumor tissues. Grouping was performed when the average tumor volume of the experimental animals reached a size suitable for grouping (approximately 200 mm³), with each group consisting of n=3-5 mice.

### [1-2: Evaluation of the Therapeutic Effect of E7820]

From the day of grouping, E7820 was administered orally at a dose of 100 mg/kg once daily to the 30 PDX model mice prepared in 1-1. Body weight and tumor volume were measured twice a week during the observation period from the day before drug administration and from the day drug administration started (day 0) to day 21. Tumor volume was measured by measuring the long and short diameters of the tumor using calipers and calculating the tumor volume according to the following formula. For animals with no detectable tumor, the tumor volume was set to 0.00 mm³.

Tumor volume (mm³)=½×long diameter (mm)×short diameter (mm)×short diameter (mm)

As a result, in 14 out of the 30 PDX model mice, the tumor volume on day 21 was smaller than on day 0, indicating a tumor reduction effect.

### [1-3: Evaluation of the Presence or Absence of Gene Mutations]

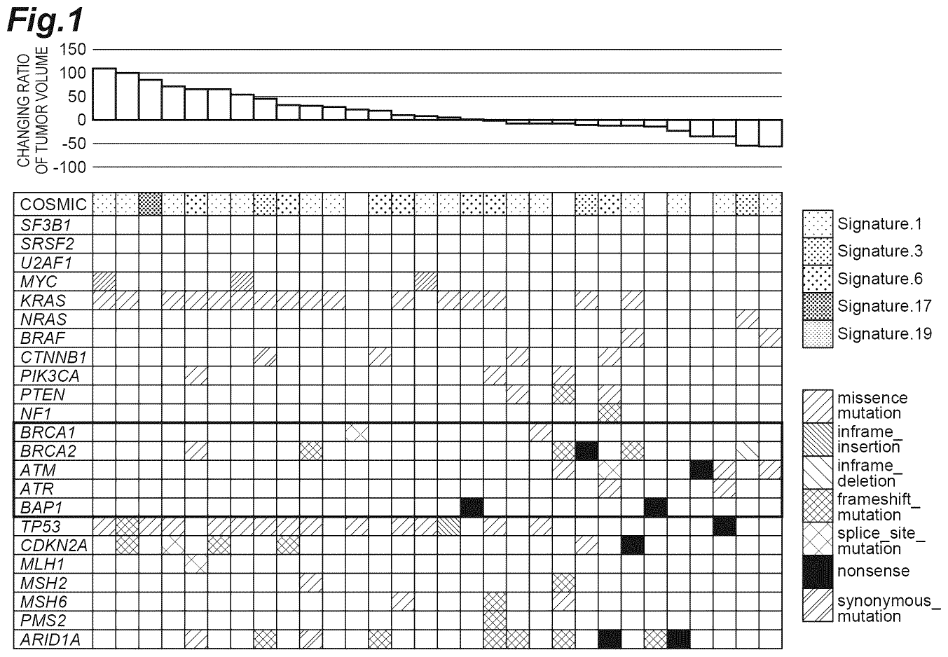

Whole exome sequencing (WES), one of the next-generation sequencing methods, was performed on genomic DNA extracted from the PDX using the NovaSeq 6000 system (Illumina), and mutation analysis was conducted. FIG. 1 shows the results of the presence or absence of various gene mutations in each mouse (lower row) when the mice are arranged in descending order based on the percentage change in tumor volume on day 21 relative to day 0 obtained in 1-2 (upper row).

As a result, among the 14 cases in which a tumor reduction effect was observed with E7820, 11 cases (79%) had mutations in one or more of the homologous recombination repair-related genes BRCA1, BRCA2, ATM, ATR, and BAP1. In contrast, among the 16 cases in which no tumor reduction effect was observed with E7820, only 3 cases (19%) had mutations in one or more of the above five genes.

### Example 2: Evaluation of the Sensitivity of Cultured Cell Lines to Cancer Therapeutics that Regulate Splicing Function Based on the Presence or Absence of Mutations and Knockouts in Homologous Recombination Repair-Related Genes

Cells from various cultured cell lines were seeded at 500 cells per well in a 96-well microplate and incubated at 37 °C under 5% CO₂. After removing the medium, a medium containing no drug, or containing 100 nM, 300 nM, or 1 μM of the drug was added, and the cells were cultured at 37 °C under 5% CO₂. The medium was replaced with fresh medium containing the same concentration of the drug on days 3, 6, and 9, with the day of drug addition being day 0. On day 10, cell viability was determined using the PrestoBlue assay.

Table 1 shows the types and origins of the cultured cell lines used, as well as the presence or absence of pathogenic mutations in homologous recombination repair-related genes evaluated in Example 1. Among the cultured cell lines, in addition to wild-type DLD-1 cells, cell lines in which the homologous recombination repair-related gene BRCA2 was knocked out (DLD-1-BRCA2 KO) and cell lines in which wild-type BRCA2 was expressed in DLD-1-BRCA2 KO (DLD-1-BRCA2 KO+BRCA2 WT) were also used.

TABLE 1

| | | Pathogenic mutation | |
|---|---|---|---|
| | | of BRCA1/BRCA2/ | |
| Cell line | Origin | ATM/BAP1 | Medium |
| BJ/TS | Fibroblast | None | DMEM |
| | | | (containing |
| | | | 10% FBS) |
| WI-38 | Fibroblast | None | DMEM |
| | | | (containing |
| | | | 10% FBS) |
| KKU-213 | Cholangiocarcinoma | None | DMEM |
| | | | (containing |
| | | | 10% FBS) |
| TFK-1 | Cholangiocarcinoma | BAP1 p.Q456* | RPMI1640 |
| | | | (containing |
| | | | 10% FBS) |
| JPDX598 | Cholangiocarcinoma | BAP1 S628* | RPMI1640 |
| | | | (containing |
| | | | 10% FBS) |
| JPDX860 | Cholangiocarcinoma | BRCA2 D426fs | RPMI1640 |
| | | | (containing |
| | | | 10% FBS) |
| JPDX919 | Cholangiocarcinoma | BAP1 Q441* | RPMI1640 |
| | | | (containing |
| | | | 10% FBS) |
| JPDX360 | Cholangiocarcinoma | None | RPMI1640 |
| | | | (containing |
| | | | 10% FBS) |
| JPDX826 | Cholangiocarcinoma | None | RPMI1640 |
| | | | (containing |
| | | | 10% FBS) |
| AU585 | Breast cancer | None | RPMI1640 |
| | | | (containing |
| | | | 10% FBS) |
| HCC1428 | Breast cancer | BRCA2 | RPMI1640 |
| | | c.4912_6841+205del | (containing |
| | | | 10% FBS) |
| HCC1937 | Breast cancer | BRCA1 | RPMI1640 |
| | | p.Q1756fs*74 | (containing |
| | | | 10% FBS) |
| NCI-H358 | Lung cancer | None | RPMI1640 |
| | | | (containing |
| | | | 10% FBS) |
| NCI-H2087 | Lung cancer | None | RPMI1640 |
| | | | (containing |
| | | | 10% FBS) |
| NCI-H1648 | Lung cancer | None | RPMI1640 |
| | | | (containing |
| | | | 10% FBS) |
| NCI-H1395 | Lung cancer | ATM p.T2666A | RPMI1640 |
| | | | (containing |
| | | | 10% FBS) |
| DLD1 | Colorectal cancer | None | RPMI1640 |
| | | | (containing |
| | | | 10% FBS) |

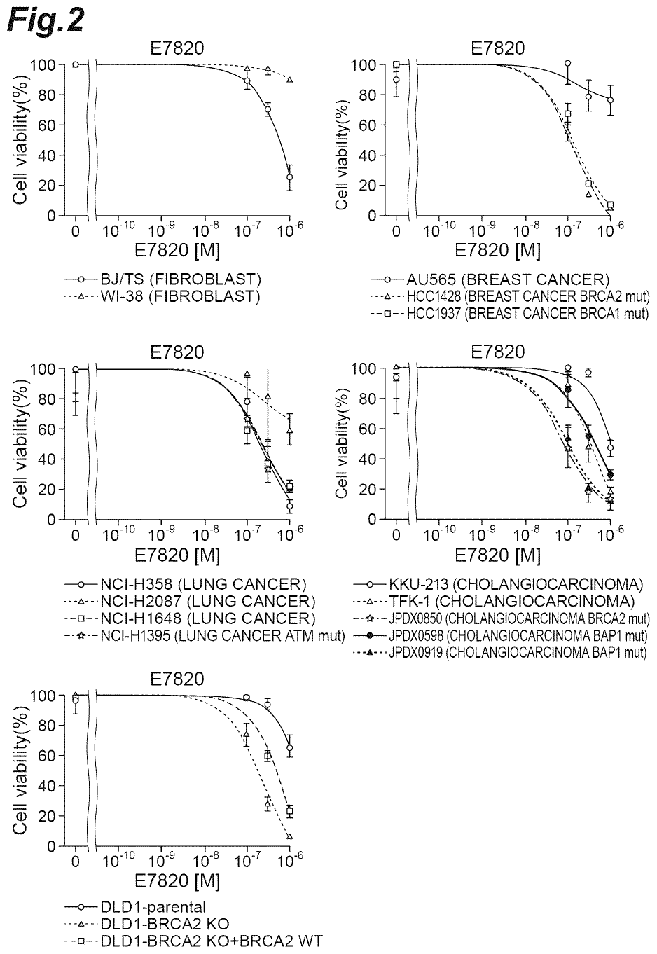

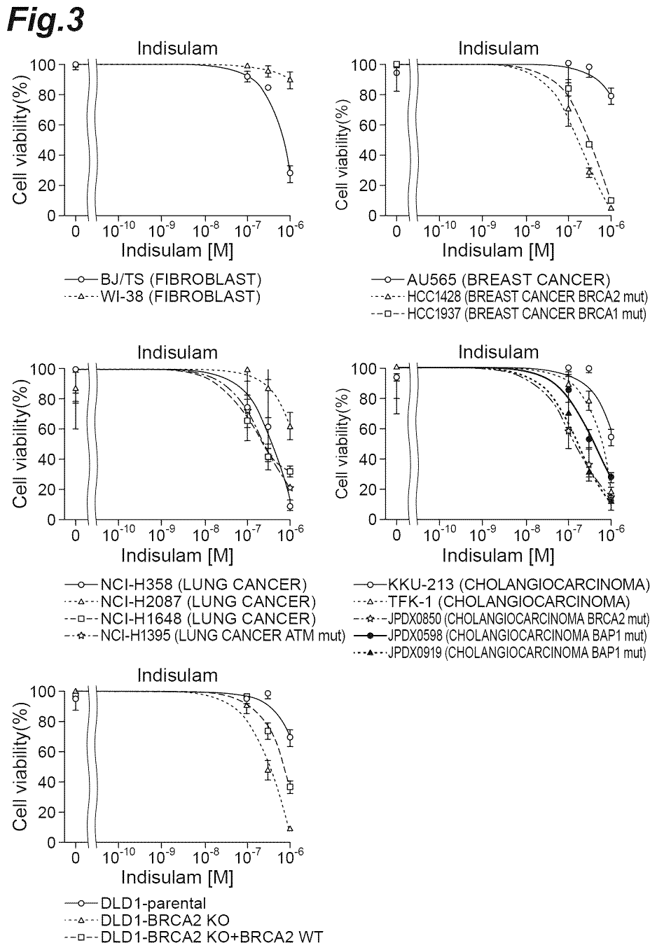

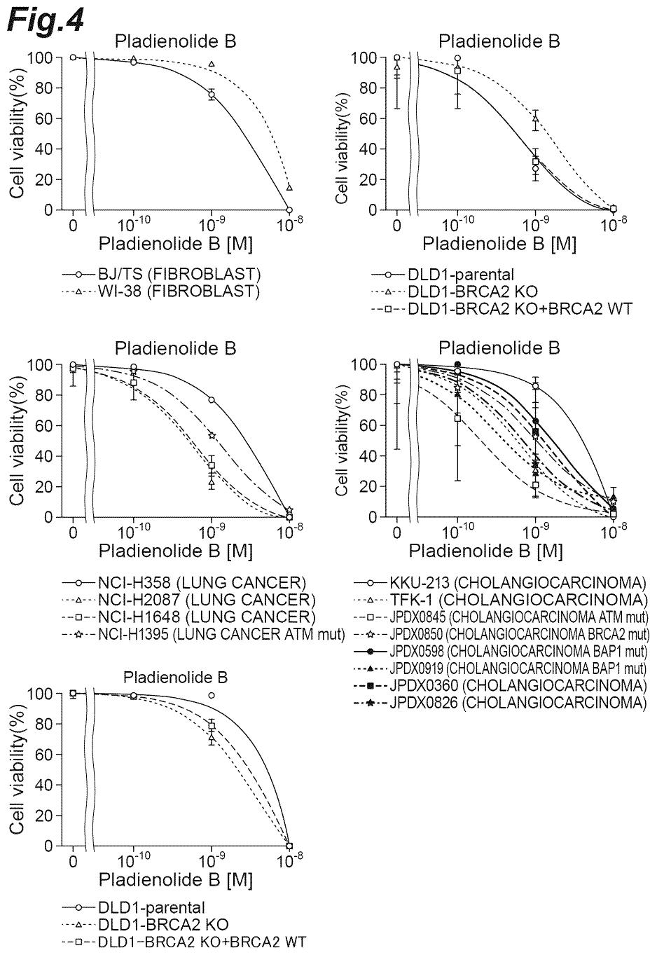

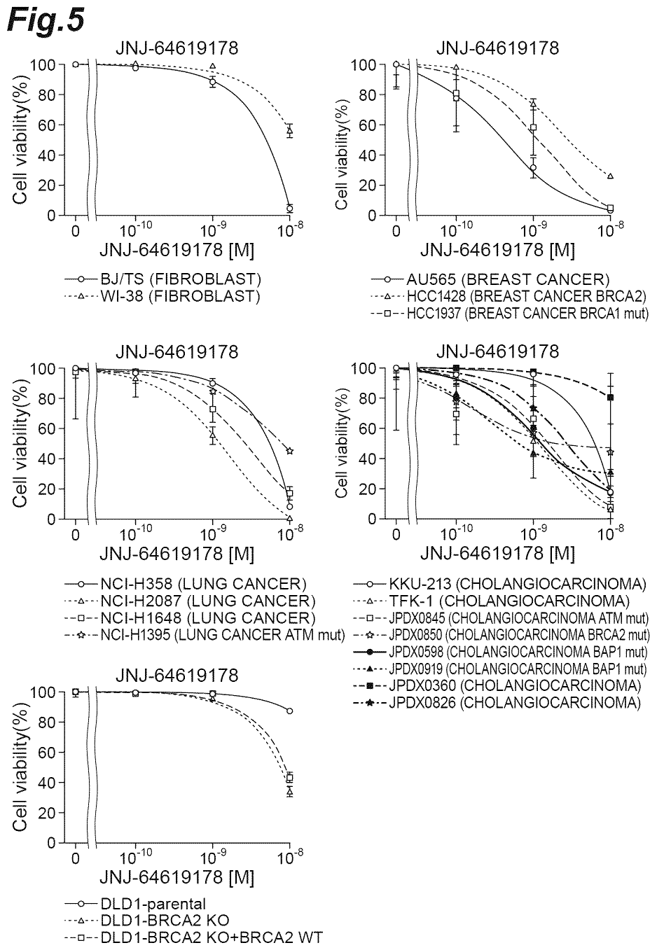

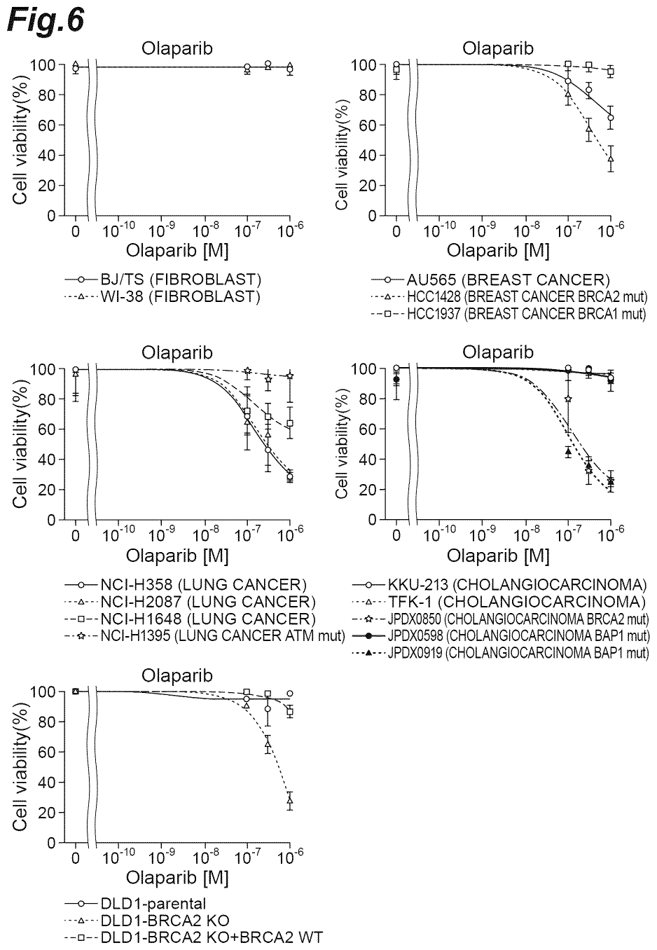

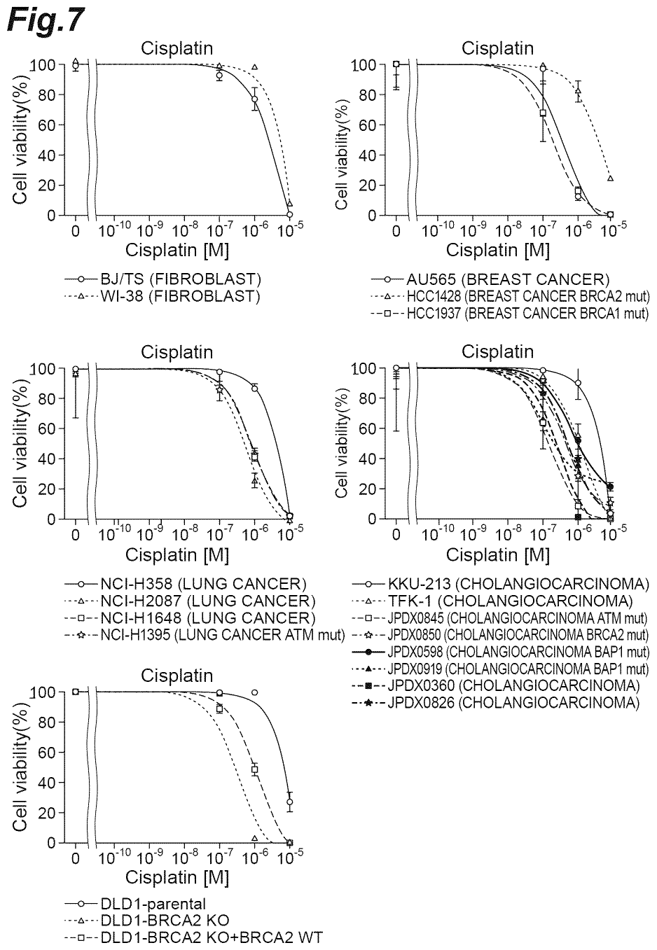

The results when using E7820, a cancer therapeutic that regulates splicing function, as the drug are shown in FIG. 2, the results when using Indisulam (E7070) are shown in FIG. 3, the results when using pladienolide B are shown in FIG. 4, and the results when using JNJ-64619178 are shown in FIG. 5. Additionally, the results when using olaparib or cisplatin, drugs known to show high sensitivity in cancer cells with mutations in homologous recombination repair-related genes, are shown in FIGS. 6 and 7, respectively.

As a result, for all drugs, there was a tendency for cell lines with mutations in homologous recombination repair-related genes to show a decrease in cell viability at low concentrations compared to cell lines without mutations, indicating that cell lines with mutations in homologous recombination repair-related genes exhibited high sensitivity to cancer therapeutics that regulate splicing function. Additionally, for all drugs, DLD-1-BRCA2 KO showed higher sensitivity compared to wild-type DLD-1, and DLD-1-BRCA2 KO+BRCA2 WT showed lower sensitivity compared to DLD-1-BRCA2 KO. These results indicate that cells with mutations in homologous recombination repair-related genes or cells with suppressed expression of homologous recombination repair-related genes, i.e., cells with decreased expression of functional homologous recombination repair-related proteins, exhibit high sensitivity to cancer therapeutics that regulate splicing function.

### Example 3: Evaluation of the Sensitivity of Cultured Cell Lines to Cancer Therapeutics that Regulate Splicing Function Based on the Presence or Absence of Knockouts and Knock-ins in Homologous Recombination Repair-Related Genes

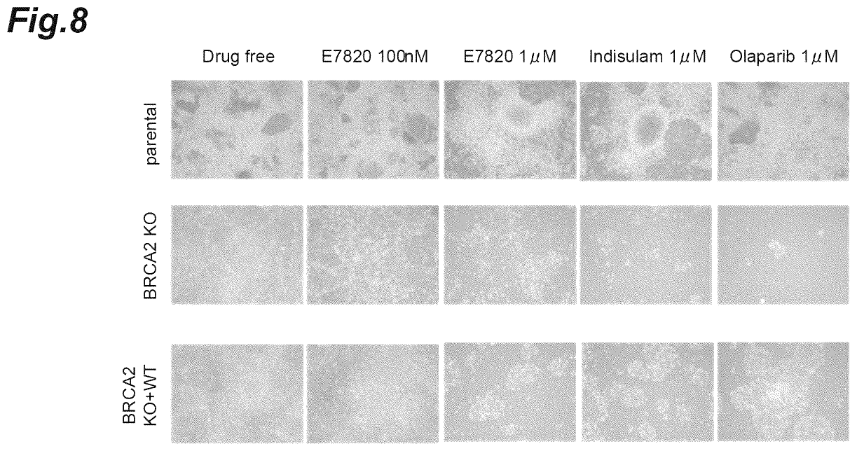

DLD-1 cells (parental), DLD-1-BRCA2 KO (BRCA2 KO), or DLD-1-BRCA2 KO+BRCA2 WT (BRCA2 KO+WT) were seeded at 3×10³ cells per well in a 6-well plate and incubated at 37 °C under 5% CO₂. After removing the medium, a medium containing no drug, or containing 100 nM E7820, 1 μM E7820, 1 μM Indisulam (E7070), or 1 μM olaparib was added, and the cells were cultured at 37 °C under 5% CO₂. The medium was replaced with fresh medium containing the same concentration of the drug on days 3, 6, and 9, with the day of drug addition being day 0. On day 12, the cells were photographed, and the sensitivity of the cells to each drug was evaluated. The results are shown in FIG. 8.

In DLD-1 cells (parental), no growth inhibitory effect was observed under any of the conditions of 100 nM E7820, 1 μM E7820, 1 pM Indisulam (E7070), or 1 μM olaparib. In DLD-1-BRCA2 KO (BRCA2 KO), significant growth inhibitory effects and induction of cell death were observed under all conditions of 1 μM E7820, 1 μM Indisulam (E7070), and 1 μM olaparib, with almost no remaining cells. On the other hand, in DLD-1-BRCA2 KO+BRCA2 WT (BRCA2 KO+WT), although growth inhibitory effects were observed under all conditions of 1 μM E7820, 1 μM Indisulam (E7070), and 1 μM olaparib, the growth of surviving cells was observed. In this experiment, olaparib served as a positive control as a drug that shows efficacy in cells with abnormal homologous recombination repair function.

### Example 4: Evaluation of the Induction of DNA Double-Strand Breaks by Cancer Therapeutics that Regulate Splicing Function Based on the Presence or Absence of Knockouts and Knock-ins in Homologous Recombination Repair-Related Genes

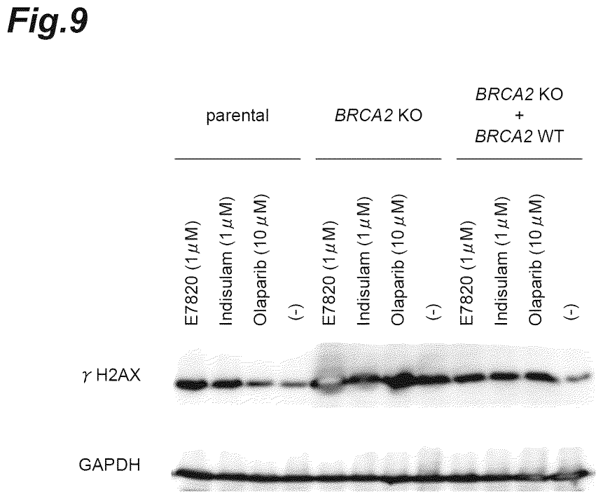

DLD-1 cells (parental), DLD-1-BRCA2 KO (BRCA2 KO), or DLD-1-BRCA2 KO+BRCA2 WT (BRCA2 KO+WT) were seeded at 1×10⁶ cells per well in a 6-well plate and incubated at 37 °C under 5% CO₂. After removing the medium, a medium containing no drug, or containing 1 μM E7820, 1 μM Indisulam (E7070), or 1 μM olaparib was added, and the cells were cultured at 37 °C under 5% CO₂ for 72 hours. After removing the medium, the cells were washed with PBS, cooled on ice, and RIPA buffer (containing protease and phosphatase inhibitors, Proteintech) was added. The cells were collected using a cell scraper. The collected samples were incubated on ice for 30 minutes, then centrifuged at 12,000×g for 15 minutes at 4 °C to pellet the cell debris. The supernatant was transferred to a new microtube without touching the pellet to obtain the lysate. The obtained lysate was used for SDS-PAGE, membrane transfer, and Western blotting to evaluate the induction of DNA double-strand breaks. The antibodies used for Western blotting were Anti-phospho-Histone H2A.X (Ser139) Antibody (anti-yH2AX antibody, cat #05-636, Sigma-Aldrich) and GAPDH (14C10) Rabbit mAb (anti-GAPDH antibody, cat #2118, Cell Signaling Technology). The results are shown in FIG. 9.

As a result, when cancer therapeutics that regulate splicing function were added to wild-type DLD-1, an increase in γH2AX levels was observed, similar to when olaparib, a known DNA damage repair inhibitor, was added, suggesting the induction of DNA double-strand breaks. Additionally, for all drugs, a more pronounced increase in yH2AX levels was observed in DLD-1-BRCA2 KO compared to wild-type DLD-1, and the extent of this increase was suppressed in DLD-1-BRCA2 KO+BRCA2 WT. These results strongly suggest that cancer therapeutics that regulate splicing function induce DNA double-strand breaks.

1-14. (canceled)

15: A method for treating cancer in a subject, comprising:
a step of determining the presence or absence of a mutation in a homologous recombination repair-related gene by using a sample taken from a subject, and
a step of administering a cancer therapeutic that regulates splicing function to the subject if one or more of the genes have the mutation.

16: The method according to claim 15, wherein the cancer therapeutic that regulates splicing function comprises one or more compounds selected from the group consisting of a RBM39 degradation inducer, a SF3b complex inhibitor, a PRMT5 inhibitor, and a type I PRMT inhibitor.

17: The method according to claim 15, wherein the cancer therapeutic that regulates splicing function comprises one or more compounds selected from the group consisting of a RBM39 degradation inducer, a SF3b complex inhibitor, and a PRMT5 inhibitor.

18: The method according to claim 15, wherein the cancer therapeutic that regulates splicing function comprises one or more compounds selected from the group consisting of E7820, E7070, NSC339004, pladienolide B, E7107, spliceostatin A, GSK3326596, PF-06939999, JNJ-64619178, and GSK3368715.

19: The method according to claim 15, wherein the homologous recombination repair-related gene comprises one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2.

20: The method according to claim 15, wherein the homologous recombination repair-related gene comprises one or more genes selected from the group consisting of BRCA1, BRCA2, ATM, ATR, and BAP1.

21: The method according to claim 15, wherein the homologous recombination repair-related gene comprises one or more genes selected from the group consisting of BRCA1 and BRCA2.

22: The method according to claim 16, wherein the homologous recombination repair-related gene comprises one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2.

23: The method according to claim 16, wherein the homologous recombination repair-related gene comprises one or more genes selected from the group consisting of BRCA1, BRCA2, ATM, ATR, and BAP1.

24: The method according to claim 16, wherein the homologous recombination repair-related gene comprises one or more genes selected from the group consisting of BRCA1 and BRCA2.

25: The method according to claim 17, wherein the homologous recombination repair-related gene comprises one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2.

26: The method according to claim 17, wherein the homologous recombination repair-related gene comprises one or more genes selected from the group consisting of BRCA1, BRCA2, ATM, ATR, and BAP1.

27: The method according to claim 17, wherein the homologous recombination repair-related gene comprises one or more genes selected from the group consisting of BRCA1 and BRCA2.

28: The method according to claim 18, wherein the homologous recombination repair-related gene comprises one or more genes selected from the group consisting of BRCA1, BRCA2, ATM, ATR, BAP1, BLM, CHEK1, CHEK2, FANCA, FANCM, MRE11A, PALB2, RAD51, RAD54, BRIP1, CDK12, and HDAC2.

29: The method according to claim 18, wherein the homologous recombination repair-related gene comprises one or more genes selected from the group consisting of BRCA1, BRCA2, ATM, ATR, and BAP1.

30: The method according to claim 18, wherein the homologous recombination repair-related gene comprises one or more genes selected from the group consisting of BRCA1 and BRCA2.
